(19) United States
(12) Patent Application Publication
(10) Pub. No.: US 2015/0336900 A1
(43) Pub. Date: Nov. 26, 2015

(54) Process for the Synthesis of Etravirine and Its Intermediates

(71) Applicant: Biophore India Pharmaceuticals PVT. Ltd., Hyderabad (IN)

(72) Inventors: Manik Reddy PULLAGURLA, Hyderabad (IN); Jagadeesh Babu RANGISETTY, Hyderabad (IN); Mecheril Valsan NANDAKUMAR, Hyderabad (IN)

(73) Assignee: Biophore India Pharmaceuticals PVT. Ltd., Hyderabad (IN)

(21) Appl. No.: 14/438,996

(22) PCT Filed: Oct. 28, 2013
(86) PCT No.: PCT/IN2013/000653

§ 371 Date: Apr. 28, 2015

(30) Foreign Application Priority Data
Oct. 29, 2012 (IN) 4485/CHE/2012

(51) Int. Cl. C07D 239/48

(52) U.S. Cl. CPC C07D 239/48

(57) ABSTRACT

The invention discloses the synthesis of Etravirine via intermediate 4-((4-amino-5-bromo-6-chloropyrimidin-2-yl) amino)benzonitrile and process for the preparation of Etravirine of the formula-I.

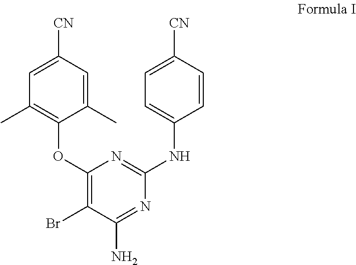 [SMILES: Cc1cc(C#N)cc(C)c1Oc1nc(Nc2ccc(C#N)cc2)nc(N)c1Br]

### FIELD OF INVENTION

The invention relates to a process for the preparation of Etravirine (formula-I) and its pharmaceutically acceptable salt for the treatment of HIV.

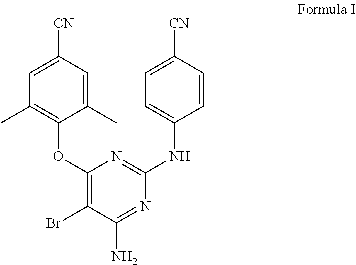 [SMILES: Cc1cc(C#N)cc(C)c1Oc1nc(Nc2ccc(C#N)cc2)nc(N)c1Br]

### BACKGROUND OF THE INVENTION

Etravirine, chemically known as 4-((6-amino-5-bromo-2-((4-cyanophenyl) amino) pyrimidin-4-yl) oxy)-3, 5-dimethylbenzonitrileis a pyrimidine derivative having Human Immunodeficiency Virus (HIV) replication inhibiting properties. It was first disclosed in U.S. Pat. No. 7,037,917 and the key step disclosed in the patent is presented in Scheme 1.

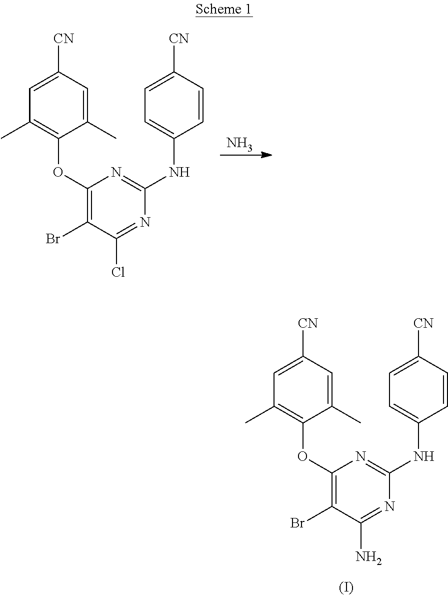

### STATE OF THE ART

The following procedures are reported for the synthesis of etravirine. U.S. Pat. No. 7,037,917 describes the synthesis of compound of formula (I) and its salt. The process for etravrine comprises reacting 4-[[6-chloro-5-bromo-2[(4-cyanophenyl) amino]-4-pyrimidinyl]oxy-3, 5-dimethylbenzonitrile with ammonia. The process requires longer reaction times and also resulted poor yields in all stages.

Bioorg. Med. Chem. Lett. (2001, 11, 2235-2239) explains a process for the synthesis of etravirine and the synthesis involes the introduction of —NH₂ group at the final stage of the synthesis. It required a high pressure and temperature for the introduction of amino group. The process also gave poor yields in the amination and phenoxide coupling reaction stages.

Organic process Research and Development (2010, 14, 657-660) describes an improved process for the synthesis of etravirine. According to the process, the synthesis starts with trichloro pyrimidine and the main aspect of this route is the introduction of bromine at the final stage of the synthesis. This method suffers from the formation of an unwanted coupling product in the aminobenzonitrile coupling stage and it needs tedious work up procedures to remove the impurity. Due the formation of the impurity, the yield of the reaction was also very low.

Process for the preparation of etravirine was described in WO 2010049952 and it describes the synthesis of etravirine through a novel. intermediate 4-((6-chloro-2-(4-cyanophenyl) amino)-4-pyrimidinyl)oxy-3,5-dimethylbenzonitrile and also reported novel crystalline forms of etravirine. In the reported synthesis, the yields obtained in some of the stages are very poor.

It is clear from the above discussion that, an efficient and cost effective method for the synthesis of etravirine is needed to overcome the difficulties associated with the known art.

### OBJECT OF THE INVENTION

The primary object of the invention is to provide a process for the preparation of Etravirine.

Another object of the invention is to provide a process for preparation of 4-((4-amino-5-bromo-6-chloropyrimidin-2-yl) amino) benzonitrileis, a key intermediate for Etravirine synthesis. A further object of the invention is to provide pharmaceutically acceptable salts of the Etravirine for the treatment of HIV.

### SUMMARY OF INVENTION

Accordingly, the inventions provides a novel process for the preparation of etravirine and 4-((4-amino-5-bromo-6-chloropyrimidin-2-yl) amino) benzonitrileis a key intermediate in the process.

The invention relates to a novel process for the synthesis of 4-((6-amino-5-bromo-2-((4-cyanophenyl) amino)pyrimidin-4-yl)oxy)-3,5-dimethylbenzonitrile, which comprises the reaction of compound VI with 4-hydroxy 3, 5-dimethyl benzonitrile.

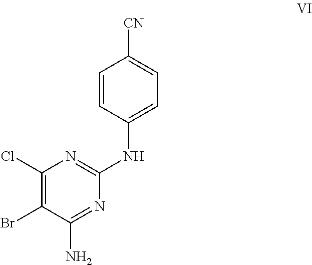 [SMILES: N#Cc1ccc(Nc2nc(N)c(Br)c(Cl)n2)cc1]

In another aspect of the invention which describes the synthesis of VI from compound V by reaction with ammonia dissolved in a suitable solvents like tetrahydrofuran, 1, 4-dioxane or dichloromethane. The ammonia reaction was carried out at 25-150° C.

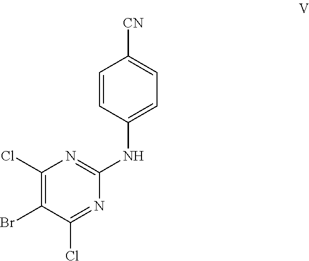 [SMILES: N#Cc1ccc(Nc2nc(Cl)c(Br)c(Cl)n2)cc1]

Compound V can be synthesized by the reaction of compound IV with a suitable brominating agent in a suitable solvents like tetrahydrofuran (THF), dichloromethane (MDC) or dimethylformamide (DMF). In another embodiment, the brominating reagent used is either bromine or N-bromosuccinimide.

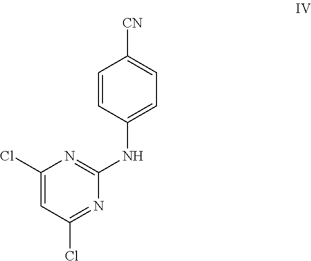 [SMILES: N#Cc1ccc(Nc2nc(Cl)cc(Cl)n2)cc1]

The synthesis of IV was achieved by reacting III with phosphorous oxychloride in presence or absence of a base at an elevated temperature. The reaction may be preferably carried out in presence or absence of a solvent and the product IV was isolated in the pure form by re-crystallization method.

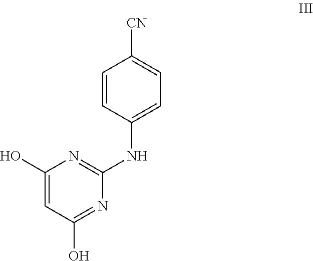 [SMILES: N#Cc1ccc(Nc2nc(O)cc(O)n2)cc1]

The compound III was prepared by the reaction of II with diethyl/dimethyl malonate using a base in presence of a suitable solvent like methanol or ethanol. The reaction is carried out in temperature range of 0-50° C.

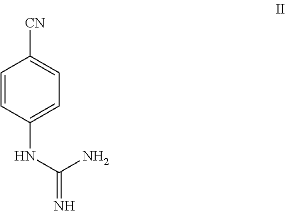 [SMILES: N#Cc1ccc(NC(=N)N)cc1]

Compound II was prepared by the reaction of 4-aminobenzonitrile with an aqueous solution of cyanamide in presence of an inorganic acid such as nitric acid, sulphuric acid or hydrochloric acid at 80-120° C. in a solvent selected from water, alcohols like methanol, ethanol or the like and the combinations thereof.

The compound of formula I may be obatined from compound of Formula VI by following the procedures described below. Examples of procedures to obtain compound of Formula VI from compound of Formula II are also provided.

### DETAILED DESCRIPTION OF THE INVENTION

We have discovered a novel process for the preparation of etravirine and 4-((4-amino-5-bromo-6-chloropyrimidin-2-yl) amino) benzonitrileis a key intermediate in the process.

The aim of the present invention is to provide an efficient process for the synthesis of etravirine to avoid all the difficulties associated with the prior art.

The invention relates to a novel process for the synthesis of 4-((6-amino-5-bromo-2-((4-cyanophenyl)amino)pyrimidin-4-yl)oxy)-3,5-dimethylbenzonitrile, which comprises the reaction of compound VI with 4-hydroxy 3, 5-dimethyl benzonitrile.

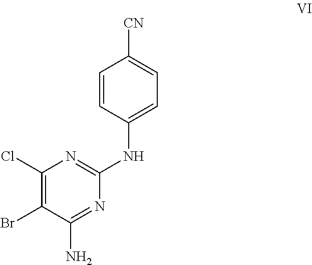 [SMILES: N#Cc1ccc(Nc2nc(N)c(Br)c(Cl)n2)cc1]

In another aspect of the invention which describes the synthesis of VI from compound V by reaction with ammonia dissolved in a suitable solvents like tetrahydrofuran, 1, 4-dioxane or dichloromethane. The ammonia reaction was carried out at 25-150° C.

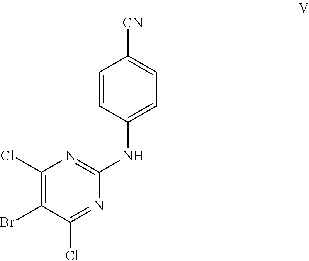 [SMILES: N#Cc1ccc(Nc2nc(Cl)c(Br)c(Cl)n2)cc1]

Compound V can be synthesized by the reaction of compound IV with a suitable brominating agent in a suitable solvents like tetrahydrofuran (THF), dichloromethane (MDC) or dimethylformamide (DMF). In another embodiment, the brominating reagent used is either bromine or N-bromosuccinimide.

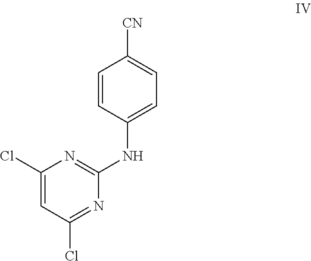 [SMILES: N#Cc1ccc(Nc2nc(Cl)cc(Cl)n2)cc1]

The synthesis of IV was achieved by reacting III with phosphorous oxychloride in presence or absence of a base at an elevated temperature. The reaction may be preferably carried out in presence or absence of a solvent and the product IV was isolated in the pure form by recrystallization method.

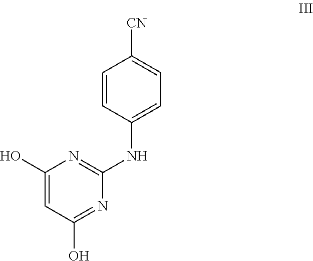 [SMILES: N#Cc1ccc(Nc2nc(O)cc(O)n2)cc1]

The compound III was prepared by the reaction of II with diethyl/dimethyl malonate using a base in presence of a suitable solvent methanol or ethanol. The reaction is carried out in temperature range of 0-50° C.

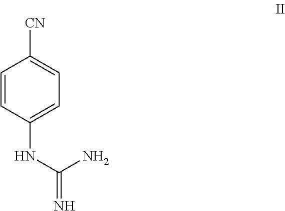 [SMILES: N#Cc1ccc(NC(=N)N)cc1]

Compound II was prepared by the reaction of 4-aminobenzonitrile with an aqueous solution of cyanamide in presence of an inorganic acid such as nitric acid, sulphuric acid or hydrochloric acid at 80-120° C. in a solvent selected from water, alcohols like methanol, ethanol or the like and the combinations thereof.

The compound of formula I may be obatined from compound of Formula VI by following the procedures described below. Examples of procedures to obtain compound of Formula VI from compound of Formula II are also provided.

### Example 1:

1-(4-cyanophenyl) guanidine(II)

100 g of 4-aminobenzonitrile was dissolved in 500 mL of methanol and cooled the reaction mixture to 10-15° C. 161 mL of con. Nitric acid was added to the reaction mixture followed by 65.6 ml of 50% aqueous solution cynamide to the reaction mixture and maintained the reaction at 65° C. for 8 hrs. Cooled the reaction mass to 0° C. and charged 500 ml of methyl-t-butyl ether at 0° C. The solids were filtered, washed with water and acetone and dried to give 30 g of the product as a solid.

### Example 2:

4-((4, 6-dihydroxypyrimidine-2-yl) amino) benzonitrile (III)

5.7 g of sodium metal was added to 200 mL of methanol at 0-5° C. with constant stirring under nitrogen atmosphere. 10 g of compound II dissolved in 200 mL of methanol was added to the solution followed by the dropwise addition of 10 g of diethyl malonate. The reaction mixture was heated to reflux for 6 h. After completion of the reaction, the solvent was removed under vacuum; water was added to the crude mixture and stirred for 30 minutes. The solid was filtered and adjusted the pH of the filtrate to 4.0-6.0. The separated solid was filtered, washed with acetone and dried to give 6.5 g of a solid.

### Example 3:

4-((4, 6-dichloropyrimidin-2-yl) amino) benzonitrile (IV)

12 g of compound III was added to 60 ml of phosphorous oxychloride and heated the reaction mixture at 90-95° C. for 12 h. After completion of the reaction, cooled the reaction mass to room temperature and 300 ml of ice water was added. The precipitated crude product was isolated by filtration. The product was purified by recrystallizing from ethyl acetate to give 6 g of the solid.

### Example 4:

4-((5-bromo-4, 6-dichloropyrimidin-2-yl) amino) benzonitrile (V)

15 g of compound IV was dissolved in 150 mL of THF at room temperature. 9 g of N-bromo succinimide was added to the reaction mixture and stirred for 24-60 h at room temperature. After completion of the reaction, the solvent was removed by distillation and the isolated material of 9 g as a solid.

### Example 5:

4-((4-amino-5-bromo-6-chloropyrimidin-2-yl) amino) benzonitrile (VI)

A sealed tube containing 100 mL of 1, 4-dioxane was purged with ammonia for 1 h at room temperature. Compound V (20 g) was added to the sealed tube and heated at 50° C. for 1 h. After completion of the reaction, the solvent was removed under vacuum and purified the compound by recrystallization using ethanol to give 11 g of the solid.

### Example-6

4-((6-amino-5-bromo-2-((4-cyanophenyl) amino) pyrimidin-4-yl) oxy)-3, 5-dimethylbenzonitrile (Etravirine, I)

16 g of compound VI was dissolved in 96 mL of N-methylpyrrolidone (NMP) at room temperature under nitrogen atmosphere. 8.4 g of potassium carbonate and 8 g of 4-hydroxy 3, 5-dimethyl benzonitrile was added to the reaction mixture and heated to 80° C. for 12 h. After completion of the reaction, cooled the reaction mixture to room temperature and water was added. The precipitated solid was isolated and dissolved in acetone by heating the solution to 50° C. The undissolved material was removed by filtration and the product was isolated by removing the solvent under vacuum. The product was further purified by recrystailization from DMF water mixture to give 10 g of the solid.

We claim:

1. A process for the preparation of Etravirine schematically represented as follows:

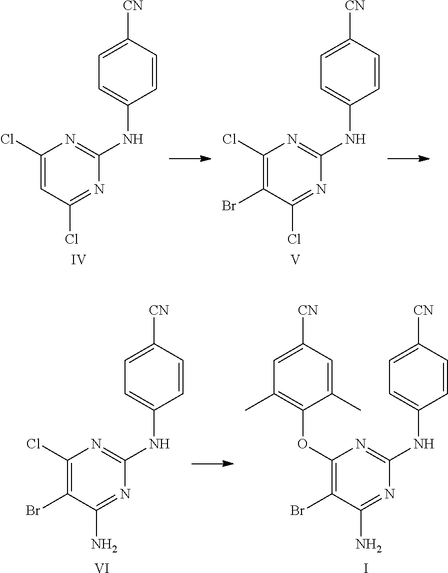

2. A process for the preparation of Etravirine which comprises the steps of:
i) brominating compound of formula IV in a solvent in the presence of N-bromosuccinimide or bromine to get compound of formula V;
ii) reaction of compound of formula V with ammonia in a solvent to give compound of formula VI;
iii) reaction of compound of formula VI with 4-hydroxy 3, 5dimethyl benzonitrile in presence of inorganic base to obtain Etravirine.

3. The process as claimed in claim 2, wherein the solvents used in step i) are selected from tetrahydrofuran, dimethylformamide and dichloromethane.

4. The process as claimed in claim 2, wherein the solvents employed in step ii) are selected from tetrahydrofuran, 1,4-dioxane and dichloromethane.

5. The process as claimed in claim 2, wherein the inorganic base employed is selected from potassium carbonate, sodium carbonate, sodium methoxide, sodium hydride, sodium hydroxide and potassium hydroxide.
